(19) United States
(12) Patent Application Publication
(10) Pub. No.: US 2022/0177704 A1
(43) Pub. Date: Jun. 9, 2022

(54) MIXTURES OF SYNTHETIC COPOLYPEPTIDE HYDROGELS

(71) Applicant: THE REGENTS OF THEUNIVERSITY OF CALIFORNIA, Oakland, CA (US)

(72) Inventors: Timothy J. Deming, Los Angele, CA (US); Yintao Sun, Los Angeles, CA (US)

(21) Appl. No.: 17/598,761

(22) PCT Filed: Mar. 27, 2020
(86) PCT No.: PCT/US20/25377

§ 371 Date: Sep. 27, 2021

Related U.S. Application Data
(60) Provisional application No. 62/824,571, filed on Mar. 27, 2019.

(51) Int. Cl. C08L 89/00; C08J 3/075; A61K 47/42

(52) U.S. Cl. CPC C08L 89/00; C08J 3/075; C08L 2205/03; C08J 2389/00; C08J 2489/00; A61K 47/42

(57) ABSTRACT

The present disclosure is directed to physical mixtures of diblock copolypeptide hydrogel (DCH) systems. These systems exhibit mechanical strength and stiffness that are synergistically increased over the individual component DCHs, to greater than would be expected for a linear combination of the components. Such systems may have utility in biomedical applications such as drug delivery.

### RELATED APPLICATIONS

This application claims the benefit of U.S. Provisional Patent Application No. 62/824,571, filed on Mar. 27, 2019. The contents of this application are hereby incorporated by reference in their entirety.

### GOVERNMENT SUPPORT

This invention was made with government support under Contract No. CHE 1807362, awarded by the National Science Foundation. The government has certain rights in the invention.

### BACKGROUND

Polyion complexes form when a pair of oppositely charged polyelectrolytes are mixed in aqueous media and phase separate as charge neutralized precipitate or coacervate complexes. However, when non-charged hydrophilic segments are connected to one or both of the polyelectrolyte chains, soluble self-assembled structures can be formed. For example, polyion complex (PIC) micelles can be formed by mixing of a pair of oppositely charged block copolymers, such as PEG-poly(L-lysine) and PEG-poly(α,β-aspartic acid) (FIG. 1A). The PIC micelles show potential as delivery vehicles for oligonucleotides, plasmid DNA, and conjugated small molecules due to their high stability, reduced immune response, and elongated blood circulation.

PIC vesicles may also be formed using similar block polymers (FIG. 1B). Similar to polypeptide based copolymer micelles, oppositely charged block copolymers with similar polyelectrolyte chain lengths produced membrane assemblies, which were shielded by exposed PEG segments in aqueous solution. These PIC vesicles do not require organic solvents for self-assembly and can provide effective stabilization and delivery of cargos, such as myoglobin. In addition, these vesicles can have pH-sensitive properties that allow tunable permeability of the membrane and intracellular release of therapeutic agents.

In additional to self-assembling into micelles and vesicles, polymers of opposite charges can also form physically crosslinked hydrogels. For example, a coacervate-based hydrogel system may be formed by mixing oppositely charged triblock copolymers, which contain central polyethyleneglycol (PEG) segments and either cationic (ammonium or guanidinium) or anionic (sulfonate or carboxylate) end blocks, in stoichiometric ratios. Due to the strong and highly efficient electrostatic crosslinking by the end blocks within the coacervate domains, high modulus hydrogels formed within seconds upon mixing at concentrations as low as 3-5 wt %. A similar polypeptide-based triblock hydrogel system has been described using PEG central segments and poly(L-glutamic acid) or poly(L-lysine) as the anionic and cationic end segments, respectively (FIG. 1C). The resulting hydrogel showed tunable mechanical strength by varying concentration, pH and polymer composition. Furthermore, it can be injected into animals, and demonstrated good biocompatibility in vivo.

There are many advantages to using block copolypeptides to form PIC-based self assemblies. The resulting materials are attractive because of their biodegradability, diverse side chain functionalities, and ability to respond to external stimuli such as pH, temperature and redox chemistry. Poly(L-methionine) can be oxidized to give water-soluble, non-ionic poly(L-methionine sulfoxide), which is being developed as a biodegradable replacement for PEG. Block copolypeptides with well-defined segment lengths may be prepared by transition metal-mediated living polymerization techniques; having well-defined structures is a desirable feature for preparing optimized self-assembling structures for biomedical applications.

Two distinct diblock copolypeptide hydrogel (DCH) systems have been described. One system is based on poly(L-methionine sulfoxide-stat-L-alanine)-block-poly(L-leucine) copolymers (i.e. M^{O}A₁₅₀L₂₀), which self-assemble in aqueous media into nonionic hydrogels (MOX-DCH) with tunable mechanical properties, excellent biocompatibility, and ability to encapsulate and release hydrophilic, hydrophobic, and live cell cargos. However, these physically cross-linked hydrogels tend to form soft hydrogels at low copolymer concentrations that also disperse over time when placed in excess aqueous media. The other recently developed DCH system was designed to incorporate oppositely charged polyionic segments that form PIC hydrogel assemblies (PIC-DCH) when mixed in aqueous media (i.e. M^{O}A₁₅₀E₅₅ and M^{O}A₁₅₀K₅₅). The PIC-DCH system retains the biocompatible properties of the MOX-DCH system, but also provides stability against dilution in aqueous media. The PIC-DCH system also allows encapsulation of hydrophilic molecules and live cells, but is less able to encapsulate hydrophobic molecules compared to the MOX-DCH system.

Therefore, there remains a need for a more robust physically cross-linked DCH system with increased mechanical stiffness, stability in media, and ability to encapsulate and release any type of molecule or cell.

### SUMMARY OF THE INVENTION

The present disclosure relates generally to physical mixtures of DCH systems and is based on the unexpected discovery that dilute physical, “dual network” mixtures of DCH systems that assemble via different processes, such as hydrophobic attraction and polyion complex formation, exhibit a synergistic increase in mechanical stiffness of the hydrogels, greater than the linear combination of the individual components. Other dual network hydrogel systems in the art are made of covalently crosslinked networks, as opposed to the purely physically crosslinked networks of the present disclosure. The dual network hydrogel systems of the present disclosure are substantially more dilute than those in the art, with concentrations of less than about 10 wt % compared to others with concentrations greater than 40 wt %. In addition, the networks of the present disclosure interpenetrate at the microscopic level, while those in the art interpenetrate at much smaller length scale (nanometers). Lastly, conventional dual network gels do not self-heal after being broken down by stress, or do so less efficiently than those of the present disclosure.

These mixed hydrogels also possess a number of beneficial features as compared to the individual components, including but not limited to self-healing, biocompatibility, resistance to dilution, ability to load hydrophilic, hydrophobic and live cell cargos, where the combined properties overcome deficiencies in each individual system. For example, if one DCH component cannot encapsulate hydrophobic cargos, and the other DCH component is not stable against dilution, the mixed gel may possess both of these properties. The dual network mixtures of DCH systems of the present disclosure have potential utility in several applications, including but not limited to cell suspension, cell culture, delivery of cells into tissues, and scaffolds for delivery of molecules and for tissue repair. Moreover, the hydrogel networks of the present disclosure may be readily prepared by 3D printing methods.

In some aspects, the present disclosure relates to a composition comprising a first copolypeptide comprising Substructure I, a second copolypeptide comprising Substructure II, a third copolypeptide comprising Substructure III, and water, wherein

Substructure I is depicted as follows:

—Xₘ—Cₚ— Substructure I;

Substructure II is depicted as follows:

—Yₙ—A_{q}— Substructure II;

Substructure III is depicted as follows:

—Zᵣ—Dₜ— Substructure III;
each instance of X is an amino acid residue independently selected from a non-ionic, hydrophilic amino acid, sarcosine, glycine, and alanine;
each instance of Y is an amino acid residue independently selected from a non-ionic, hydrophilic amino acid, sarcosine, glycine, and alanine;
each instance of Z is an amino acid residue independently selected from a non-ionic, hydrophilic amino acid, sarcosine, glycine, and alanine;
in at least 20% of the instances of C, C is an amino acid residue independently selected from a cationic, hydrophilic amino acid;
in at least 20% of the instances of A, A is an amino acid residue independently selected from an anionic, hydrophilic amino acid;
in at least 20% of the instances of D, D is an amino acid residue independently selected from a non-ionic, hydrophobic amino acid;
m is about 100 to about 600;
n is about 100 to about 600;
r is about 100 to about 600;
p is about 20 to about 100;
q is about 20 to about 100;
t is about 10 to about 100;
at least 90 mol % of the C amino acid residues are (d)-amino acid residues or at least 90 mol % of the C amino acid residues are (1)-amino acid residues;
at least 90 mol % of the A amino acid residues are (d)-amino acid residues or at least 90 mol % of the A amino acid residues are (1)-amino acid residues; and
at least 90 mol % of the D amino acid residues are (d)-amino acid residues or at least 90 mol % of the D amino acid residues are (1)-amino acid residues;
the first copolypeptide and the second copolypeptide are not covalently linked to the third copolypeptide;
the total concentration of the first copolypeptide and the second copolypeptide is about 1% to about 15%, such as about 1% to about 10%, preferably about 5.0 wt. %; and
the concentration of the third copolypeptide is about 1% to about 10%, such as about 1% to about 5%, preferably about 2.5 wt. %.

### BRIEF DESCRIPTION OF THE DRAWINGS

FIG. 1 is a schematic representation of a PIC hydrogel forming a micelle.

FIGS. 2, 3A and 3B show mechanical properties of exemplary diblock copolypeptide hydrogels according to some aspects of the present disclosure.

FIGS. 4A and 4B show rheological measurements for exemplary diblock copolypeptide hydrogels according to some aspects of the present disclosure.

FIGS. 5A-5C show stability against dilution for exemplary diblock copolypeptide hydrogels according to some aspects of the present disclosure.

FIG. 6 shows normalized swelling ratio measurements in exemplary diblock copolypeptide hydrogel compositions according to some aspects of the present disclosure.

FIGS. 7 and 8 show mechanical recovery of exemplary diblock copolypeptide hydrogels according to some aspects of the present disclosure.

FIGS. 9A-9F show laser scanning confocal microscopy images of an exemplary fluorescently labeled diblock copolypeptide hydrogel according to some aspects of the present disclosure.

### DETAILED DESCRIPTION

Protein and peptide based hydrogels are used for many applications, ranging from personal care products, food and cosmetic thickeners to support matrices for drug delivery and tissue replacement. The polyion complex DCH compositions described here offer many advantages over most other hydrogels since many molecular variables can be varied to adjust their physical properties. While the stiffness of most hydrogels is mainly adjusted either by varying polymer concentration or crosslink density, DCH stiffness can also be tuned by these parameters, or by altering amino acid composition, hydrophilic to hydrophobic ratio, molecular weight, or block architecture of the polymers. This ability to tune properties in different ways offers a facile means to adjust gel stiffness independently of concentration or crosslink density by altering the stiffness of scaffold fibrils.

The ability to control nanoscale and bulk properties by molecular design, combined with DCH injectability and abundant sites for functionalization, also makes DCH innovative candidates for use as biomaterials. DCH are unique biomaterials in that they are able to form hydrogels at low concentrations in water (<10 wt %), are fully synthetic, are composed entirely of amino acids connected by natural peptide bonds, are biodegradable, and their amphiphilic nature allows them to serve as effective carriers for delivery of both hydrophilic and hydrophobic molecules. DCH can also be injected through small-bore cannulae, after which they rapidly re-assemble into rigid gel networks allowing for minimally invasive delivery. The combination of all these properties makes ionic DCH a promising synthetic biomaterial for experimental investigations in vitro and in vivo, and potentially useful in therapeutic strategies for treatment of medical disorders or injury.

Due to minimal toxicity, polyion complex DCH_{PIC} exhibit numerous advantageous properties over ionic DCH, or other biomaterials, for use in in vitro cell culture and in vivo delivery of cells, either alone or in combination with hydrophilic and hydrophobic molecules encapsulated within the gels, for both as tools for experimental investigations and for potential therapeutic strategies. Example potential areas for their use are as depots for local delivery of therapeutics in chronic wounds, for use in prevention/treatment of STDs and HIV infections, for applications in the eyes or lungs, in the brain for treatment of glioblastoma multiforme, or for more general local delivery in tumors. Other potential uses are for cell expansion/cell culture in vitro, drug testing in 3D in vitro cell cultures, or for grafting cells in vivo, such as delivery of neural stem cells into the central nervous system.

In some aspects, the present disclosure relates to a composition comprising a first copolypeptide comprising Substructure I, a second copolypeptide comprising Substructure II, a third copolypeptide comprising Substructure III, and water, wherein

Substructure I is depicted as follows:

—Xₘ—Cₚ— Substructure I;

Substructure II is depicted as follows:

—Yₙ—A_{q}— Substructure II;

Substructure III is depicted as follows:

—Zᵣ—Dₜ— Substructure III;
each instance of X is an amino acid residue independently selected from a non-ionic, hydrophilic amino acid, sarcosine, glycine, and alanine;
each instance of Y is an amino acid residue independently selected from a non-ionic, hydrophilic amino acid, sarcosine, glycine, and alanine;
each instance of Z is an amino acid residue independently selected from a non-ionic, hydrophilic amino acid, sarcosine, glycine, and alanine;
in at least 20% of the instances of C, C is an amino acid residue independently selected from a cationic, hydrophilic amino acid;
in at least 20% of the instances of A, A is an amino acid residue independently selected from an anionic, hydrophilic amino acid;
in at least 20% of the instances of D, D is an amino acid residue independently selected from a non-ionic, hydrophobic amino acid;
m is about 100 to about 600;
n is about 100 to about 600;
r is about 100 to about 600;
p is about 20 to about 200;
q is about 20 to about 200;
t is about 10 to about 200;
at least 90 mol % of the C amino acid residues are (D)-amino acid residues or at least 90 mol % of the C amino acid residues are (L)-amino acid residues;
at least 90 mol % of the A amino acid residues are (D)-amino acid residues or at least 90 mol % of the A amino acid residues are (L)-amino acid residues; and
at least 90 mol % of the D amino acid residues are (D)-amino acid residues or at least 90 mol % of the D amino acid residues are (L)-amino acid residues;
the first copolypeptide and the second copolypeptide are not covalently linked to the third copolypeptide;
the total concentration of the first copolypeptide and the second copolypeptide is about 1% to about 15%; and
the concentration of the third copolypeptide is about 1% to about 10%.

In some aspects, the present disclosure relates to a composition comprising a first copolypeptide comprising Substructure I′, a second copolypeptide comprising Substructure II′, a third copolypeptide comprising Substructure III′, and water, wherein

Substructure I is depicted as follows:

—Xₘ—Cₚ— Substructure I′;

Substructure II is depicted as follows:

—Yₙ—A_{q}— Substructure II′;

Substructure III is depicted as follows:

—Zᵣ—Dₜ— Substructure III′;

each instance of X is an amino acid residue independently selected from a non-ionic, hydrophilic amino acid, glycine, and alanine;

each instance of Y is an amino acid residue independently selected from a non-ionic, hydrophilic amino acid, glycine, and alanine;

each instance of Z is an amino acid residue independently selected from a non-ionic, hydrophilic amino acid, glycine, and alanine;

in at least 20% of the instances of C, C is an amino acid residue independently selected from a cationic, hydrophilic amino acid, or a salt thereof;

in at least 20% of the instances of A, A is an amino acid residue independently selected from an anionic, hydrophilic amino acid, or a salt thereof;

in at least 20% of the instances of D, D is an amino acid residue independently selected from a non-ionic, hydrophobic amino acid;

m is about 100 to about 600;

n is about 100 to about 600;

r is about 100 to about 600;

p is about 20 to about 100;

q is about 20 to about 100;

t is about 10 to about 100;

at least 90 mol % of the C amino acid residues are (D)-amino acid residues or at least 90 mol % of the C amino acid residues are (L)-amino acid residues;

at least 90 mol % of the A amino acid residues are (D)-amino acid residues or at least 90 mol % of the A amino acid residues are (L)-amino acid residues; and

at least 90 mol % of the D amino acid residues are (D)-amino acid residues or at least 90 mol % of the D amino acid residues are (L)-amino acid residues;

the first copolypeptide and the second copolypeptide are not covalently linked to the third copolypeptide;

the total concentration of the first copolypeptide and the second copolypeptide is about 1% to about 15%, such as about 1% to about 10%, preferably about 5.0 wt. %; and

the concentration of the third copolypeptide is about 1% to about 10%, such as about 1% to about 5%, preferably about 2.5 wt. %.

In certain embodiments, each instance of X is an amino acid residue independently selected from a non-ionic, hydrophilic amino acid. In certain embodiments, each instance of X is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, each instance of X is an amino acid residue independently selected from methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, at least 90 mol % of the X amino acid residues are (D)-amino acid residues. In certain embodiments, at least 85 mol % of the X amino acid residues are methionine sulfoxide. In certain preferred embodiments, at least 85 mol % of the X amino acid residues are methionine sulfoxide, and the remaining X amino acid residues are alanine. In even further preferred embodiments, about 88 mol % of the X amino acid residues are methionine sulfoxide, and about 12 mol % of the X amino acid residues are alanine.

In certain embodiments, Y is an amino acid residue independently selected from a non-ionic, hydrophilic amino acid. In certain embodiments, each instance of Y is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, each instance of Y is an amino acid residue independently selected from methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide. In certain embodiments, at least 90 mol % of the Y amino acid residues are (D)-amino acid residues. In other embodiments, at least 90% of the Y amino acid residues are (L)-amino acid residues. In certain embodiments, at least 85 mol % of the Y amino acid residues are methionine sulfoxide. In certain preferred embodiments, at least 85 mol % of the Y amino acid residues are methionine sulfoxide, and the remaining Y amino acid residues are alanine. In even further preferred embodiments, about 88 mol % of the Y amino acid residues are methionine sulfoxide, and about 12 mol % of the Y amino acid residues are alanine.

In certain embodiments, each instance of C is an amino acid residue independently selected from a cationic, hydrophilic amino acid, or a salt thereof. In certain embodiments, at least 90% of the C amino acid residues are (D)-amino acid residues. In other embodiments, at least 90% of the C amino acid residues are (L)-amino acid residues. In certain embodiments, each instance of C is lysine, ornithine, or arginine. In certain preferred embodiments, each instance of C is (L)-lysine. In other preferred embodiments, each instance of C is (D)-lysine.

In certain embodiments, each instance of A is an amino acid residue independently selected from an anionic, hydrophilic amino acid, or a salt thereof. In certain embodiments, at least 90% of the A amino acid residues are (D)-amino acid residues. In other embodiments, at least 90% of the A amino acid residues are (L)-amino acid residues. In certain embodiments, each instance of A is glutamic acid or aspartic acid. In certain preferred embodiments, each instance of A is (L)-glutamic acid. In other preferred embodiments, A is (D)-glutamic acid.

In certain embodiments, each instance of Z is an amino acid residue independently selected from a non-ionic, hydrophilic amino acid. In certain embodiments, each instance of Z is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, homomethionine sulfoxide. In certain embodiments, each instance of Z is an amino acid residue independently selected from methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, homomethionine sulfoxide. In certain embodiments, at least 90 mol % of the Z amino acid residues are (D)-amino acid residues. In other embodiments, at least 90 mol % of the Z amino acid residues are (L)-amino acid residues. In certain embodiments, at least 85 mol % of the Z amino acid residues are methionine sulfoxide. In certain embodiments, at least 85 mol % of the Z amino acid residues are methionine sulfoxide, and the remaining Z amino acid residues are alanine. In certain preferred embodiments, at least 85 mol % of the Z amino acid residues are methionine sulfoxide, and the remaining Z amino acid residues are alanine. In certain even further preferred embodiments, about 88 mol % of the Z amino acid residues are methionine sulfoxide, and about 12 mol % of the Z amino acid residues are alanine.

In certain embodiments, each instance of D is an amino acid residue independently selected from a non-ionic, hydrophobic amino acid. In certain embodiments, at least 90% of the D amino acid residues are (D)-amino acid residues. In other embodiments, at least 90% of the D amino acid residues are (L)-amino acid residues. In certain embodiments, each instance of D is leucine, alanine, or phenylalanine. In certain preferred embodiments, each instance of D is (L)-leucine. In other preferred embodiments, each instance of D is (D)-leucine.

In certain embodiments, m is about 100, about 110, about 120, about 130, about 140, about 150, about 160, about 170, about 180, about 190, about 200, about 210, or about 220. In certain preferred embodiments, m is about 120, about 130, about 140, about 150, about 160, about 170, about 180, or about 190.

In certain embodiments, n is about 100, about 110, about 120, about 130, about 140, about 150, about 160, about 170, about 180, about 190, about 200, about 210, or about 220. In certain preferred embodiments, n is about 120, about 130, about 140, about 150, about 160, about 170, about 180, or about 190.

In certain embodiments, r is about 100, about 110, about 120, about 130, about 140, about 150, about 160, about 170, about 180, about 190, about 200, about 210, or about 220. In certain preferred embodiments, r is about 120, about 130, about 140, about 150, about 160, about 170, about 180, or about 190.

In certain embodiments, p is about 20, about 30, about 40, about 50, about 60, about 70, about 80, about 90, or about 100.

In certain embodiments, q is about 20, about 30, about 40, about 50, about 60, about 70, about 80, about 90, or about 100.

In certain embodiments, t is about 10, about 20, about 30, about 40, about 50, about 60, about 70, about 80, about 90, or about 100.

In certain embodiments, the polydispersity of the first copolypeptide is less than 1.5. In certain embodiments, the polydispersity of the second copolypeptide is less than 1.5.

In certain embodiments, the number of amino acid residues in the first copolypeptide is from about 90% to about 110% of the number of amino acid residues in the second copolypeptide.

In certain embodiments, the composition comprises (M^{O}A)₁₅₅E₃₀, (M^{O}A)₁₅₅E₆₀, (M^{O}A)₁₅₅E₉₀, (M^{O}A)₁₅₅E₁₂₀, (M^{O}A)₁₅₅(rac-E)₆₀, (M^{O}A)₁₅₅K₃₀, (M^{O}A)₁₅₅K₆₀, (M^{O}A)₁₅₅K₉₀, (M^{O}A)₁₅₅K₁₂₀, (M^{O}A)₁₅₀E₅₅, (M^{O}A)₁₅₀K₅₅, or (M^{O}A)₁₅₀L₂₀, or a combination of the foregoing.

In certain embodiments, the composition comprises (M^{O}A)₁₅₀E₅₅, (M^{O}A)₁₅₀K₅₅, or (M^{O}A)₁₅₀L₃₀, or a combination thereof.

In certain embodiments, the composition comprises (M^{O}A)₁₅₀K₅₅ and (M^{O}A)₁₅₀L₂₀.

In certain embodiments, the composition comprises (M^{O}A)₁₅₀E₅₅ and (M^{O}A)₁₅₀L₃₀.

In certain embodiments, the composition comprises (M^{O}A)₁₅₀E₅₅, (M^{O}A)₁₅₀K₅₅, and (M^{O}A)₁₅₀L₃₀.

In certain embodiments, the concentration of the third copolypeptide is about 1% to about 5%. In certain embodiments, the concentration of the third copolypeptide in the composition is about 2.5 wt. %. In certain embodiments, the total concentration of the first copolypeptide and the second copolypeptide is about 1% to about 10%. In certain embodiments, the total concentration of the first copolypeptide and the second copolypeptide in the composition is about 5.0 wt. %. In certain embodiments, the total concentration of the first copolypeptide and the second copolypeptide in the composition is about 5.0 wt. %, and the concentration of the third copolypeptide in the composition is about 2.5 wt. %. In certain embodiments, the total concentration of the first copolypeptide and the second copolypeptide in the composition is about 5.0 wt. %.

In certain embodiments, the molar ratio of C to A is from about 0.95 to about 1.05. In certain embodiments, the molar ratio of X to Y is from about 0.95 to about 1.05. In certain embodiments, the molar ratio of D to A is from about 0.4 to about 0.6.

In certain embodiments, the composition further comprises a salt. In certain embodiments, the concentration of the salt in the composition is less than about 500 mM. In certain embodiments, the concentration of the salt in the composition is from about 100 mM to about 300 mM. In certain preferred embodiments, the salt is NaCl.

In certain embodiments, the composition further comprises a buffer.

In certain embodiments, the composition further comprises a plurality of cells.

In other aspects, the present disclosure relates to a method of making compositions disclosed herein, the method comprising: dissolving the first copolypeptide in an aqueous medium; separately adding the third copolypeptide to the aqueous medium to form a mixture;

and mixing the mixture with a solution of the second copolypeptide, thereby forming the composition. In certain embodiments, the aqueous medium further comprises an alcohol selected from methanol, ethanol, and isopropanol. In certain embodiments, the alcohol is methanol. In certain preferred embodiments, the aqueous medium comprises about 30% to about 70% methanol. In certain even further preferred embodiments, the aqueous medium comprises about 50% methanol. In certain preferred embodiments, the mixing is rapid mixing, such as vortexing.

In still other aspects, the present disclosure relates to a method of making compositions disclosed herein, the method comprising: dissolving the second copolypeptide in an aqueous medium; separately adding the third copolypeptide to the aqueous medium to form a mixture; and mixing the mixture with a solution of the first copolypeptide, thereby forming the composition. In certain embodiments, the aqueous medium further comprises an alcohol selected from methanol, ethanol, and isopropanol. In certain embodiments, the alcohol is methanol. In certain preferred embodiments, the aqueous medium comprises about 30% to about 70% methanol. In certain even further preferred embodiments, the aqueous medium comprises about 50% methanol. In certain preferred embodiments, the mixing is rapid mixing, such as vortexing.

In yet other aspects, the present disclosure relates to a method of delivering a drug to a biological target using a composition disclosed herein, the method comprising: dissolving the drug in a first aqueous medium; dissolving the first copolypeptide in the first aqueous medium to form a second aqueous medium; separately adding the third copolypeptide to the second aqueous medium to form a mixture; mixing the mixture with a solution of the second copolypeptide, thereby forming the composition encapsulating the drug; and contacting the biological target with the composition. In certain embodiments, the biological target is a cell, organ, tissue, or protein. In certain embodiments, the drug is hydrophobic. In certain embodiments, the drug is a chemotherapeutic agent. In certain embodiments, the drug is anthracycline. In certain embodiments, the drug is doxorubicin. In other embodiments, the drug is a hydrophilic drug. In certain embodiments, the hydrophilic drug is a protein or an antibody. In certain embodiments, the aqueous medium comprises an alcohol selected from methanol, ethanol, and isopropanol. In certain preferred embodiments, the alcohol is methanol.

In yet other aspects, the present disclosure relates to a method of delivering a drug to a biological target using a composition disclosed herein, the method comprising: dissolving the drug in a first aqueous medium; dissolving the second copolypeptide in the first aqueous medium to form a second aqueous medium; separately adding the third copolypeptide to the second aqueous medium to form a mixture; mixing the mixture with a solution of the first copolypeptide, thereby forming the composition encapsulating the drug; and contacting the biological target with the composition. In certain embodiments, the biological target is a cell, organ, tissue, or protein. In certain embodiments, the drug is hydrophobic. In certain embodiments, the drug is a chemotherapeutic agent. In certain embodiments, the drug is anthracycline. In certain embodiments, the drug is doxorubicin. In other embodiments, the drug is a hydrophilic drug. In certain embodiments, the hydrophilic drug is a protein or an antibody. In certain embodiments, the aqueous medium comprises an alcohol selected from methanol, ethanol, and isopropanol. In certain preferred embodiments, the alcohol is methanol.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The definition of each expression, e.g., alkyl, m, n, and the like, when it occurs more than once in any structure, is intended to be independent of its definition elsewhere in the same structure.

Certain compounds contained in compositions of the invention may exist in particular geometric or stereoisomeric forms. In addition, polymers of the invention may also be optically active. The invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

For purposes of this invention, the chemical elements are identified in accordance with the Periodic Table of the Elements, CAS version, Handbook of Chemistry and Physics, 67th Ed., 1986-87, inside cover.

The term “mixing” refers to any method of contacting one component of a mixture with another component of a mixture, including agitating, blending, combining, contacting, milling, shaking, sonicating, spraying, stirring, and vortexing.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

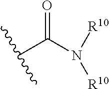

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

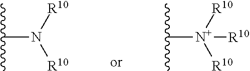

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

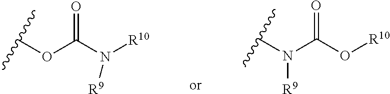

wherein R⁹ and R¹⁰ independently represent hydrogen or a hydrocarbyl group, such as an alkyl group, or R⁹ and R¹⁰ taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical.

Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “heteroalkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. Illustrative substituents include, for example, those described herein above. This invention is not intended to be limited in any manner by the permissible substituents of organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

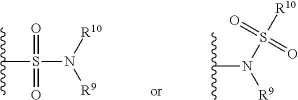

wherein R⁹ and R¹⁰ independently represents hydrogen or hydrocarbyl, such as alkyl, or R⁹ and R¹⁰ taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

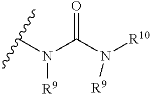

wherein R⁹ and R¹⁰ independently represent hydrogen or a hydrocarbyl, such as alkyl, or either occurrence of R⁹ taken together with R¹⁰ and the intervening atom(s) complete a heterocycle having from 4 to 8 atoms in the ring structure.

### EXEMPLIFICATION

The syntheses of PIC-DCH and MOX-DCH have been previously described, for example, in International Patent Application No. PCT/US2018/053050, filed Sep. 27, 2018, and in International Patent Application Publication No. WO 2014/134203, published Sep. 4, 2014, the disclosures of which are incorporated by reference herein in their entirety.

### Example 1: General Materials and Instrumentation

Tetrahydrofuran (THF), hexanes, and methylene chloride were dried by purging with nitrogen and passage through activated alumina columns prior to use. Co(PMe₃)₄ and amino acid N-carboxyanhydride (NCA) monomers were prepared according to literature procedures.¹ All other chemicals were purchased from commercial suppliers and used without further purification unless otherwise noted. Selecto silica gel 60 (particle size 0.032-0.063 mm) was used for flash column chromatography. Fourier transform infrared (FTIR) spectra were acquired on a Perkin Elmer RX1 FTIR spectrophotometer calibrated using polystyrene film, and attenuated total reflectance infrared (ATR-IR) data were collected using a Perkin Elmer Spectrum 100 FTIR spectrometer equipped with a universal ATR sample accessory. ¹H NMR spectra were acquired on a Bruker ARX 400 spectrometer. Tandem gel permeation chromatography/light scattering (GPC/LS) was performed using an SSI Accuflow Series III pump equipped with Wyatt DAWN EOS light scattering and Optilab REX refractive index detectors. Separations were achieved using 100 Å and 1000 Å PSS-PFG 7 μm columns at 30° C. with 0.5% (w/w) KTFA in 1,1,1,3,3,3-hexafluoroisopropanol (HFIP) as eluent and sample concentrations of 10 mg/ml. Pyrogen free deionized (DI) water was obtained from a Millipore Milli-Q Biocel A10 purification unit.

### Example 2: Copolypeptide Synthesis

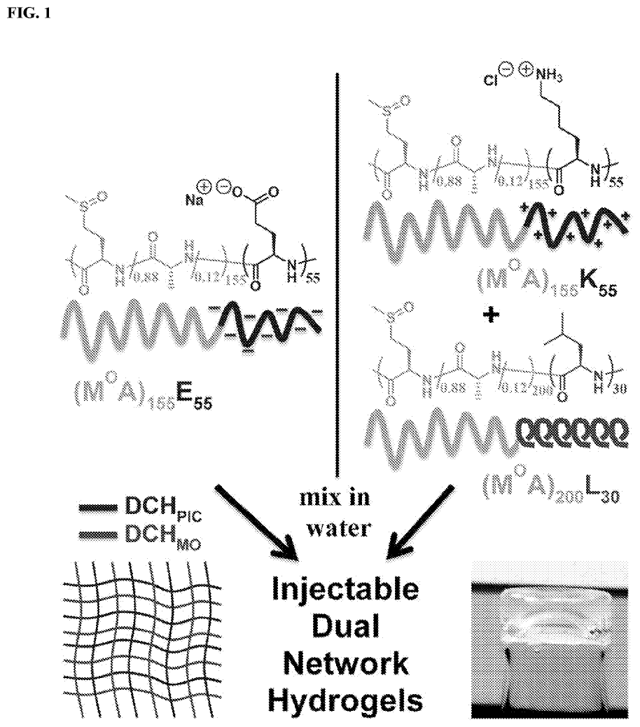

FIG. 1 shows a schematic of preparation of diblock copolypeptide dual network hydrogels, DCH_{DN}, via combination of (M^{O}A)₁₅₅E/K₅₅ (DCH_{PIC}) and (M^{O}A)₂₀₀L₃₀ (DCH_{MO}) that assemble into discrete physical networks via polyion complex and hydrophobic interactions, respectively.

Samples of poly(L-methionine sulfoxide_{0.88}-stat-L-alanine_{0.12})₁₅₅-block-poly(L-lysine)₅₅, (M^{O}A)₁₅₅K₅₅, poly(L-methionine sulfoxide_{0.88}-stat-L-alanine_{0.12})₁₅₅-block-poly(L-glutamate)₅₅, (M^{O}A)₁₅₅E₅₅, and poly(L-methionine sulfoxide_{0.88}-stat-L-alanine_{0.12})₂₀₀-block-poly(L-leucine)₃₀ were prepared as previously described.^{2,3} In general, all polymerization reactions were performed in an N₂ filled glove box using anhydrous solvents. To a solution of L-methionine NCA (Met NCA) and L-alanine NCA (Ala NCA) in THF (50 mg/ml) was added a solution of Co(PMe₃)₄ in THF (20 mg/ml). Reactions were let to stir at ambient temperature (ca. 22° C.) for 60 min. Complete consumption of NCA was confirmed by FTIR spectroscopy, and then the desired amount of γ-benzyl-L-glutamate NCA (Bn-Glu NCA), c-trifluoroacetyl-L-lysine NCA (TFA-Lys NCA) or L-leucine NCA in THF (50 mg/ml) was added to the reaction mixtures, which were let to stir for an additional 60 min. FTIR was used to confirm complete consumption of all NCAs. Once polymerizations were completed the block copolypeptide solutions were removed from the glove box, precipitated into 10 mM HCl (20 ml), and then washed with 10 mM aqueous HCl (2×20 ml) to remove residual cobalt ions. The white precipitates were then washed with DI water (3×20 ml) and freeze-dried to give products as white solids. Subsequent oxidation of samples, followed by deprotection of Bn-Glu or TFA-Lys groups were performed as previously described.

TABLE 1
Characterization data for diblock copolypeptides.

| Sample | M_{w}/Mₙ^{a} | Composition^{b} | Yield (%)^{c} |
|---|---|---|---|
| (M^{O} A)₁₅₅E₅₅ | 1.37 | (M0A)152E52 | 92 |
| (M^{O} A)₁₅₅K₅₅ | 1.38 | (M0A)152K54 | 94 |
| (M^{O} A)₂₀₀L₃₀ | 1.40 | (M0A)215L31 | 91 |

^{a}Dispersity of oxidized, protected block copolypeptides
were determined by GPC/LS.
^{b}Actual amino acid compositions of oxidized, deprotected block
copolypeptides were determined by ¹H NMR integrations. Degree of
polymerization of initial (MA)ₓ segments was determined by
end-group analysis using ¹H NMR.
^{c}Total isolated yield of deprotected, purified block copolypeptides.

Preparation of (M^{O}A)₁₅₅E/K₅₅+(M^{O}A)₂₀₀L₃₀ dual network hydrogels (DCH_{DN})

The cationic diblock copolypeptide, M^{O}A₁₅₀K₅₅, was first dissolved in 1×PBS at the desired concentration (e.g. 5 wt %). This solution was then used to dissolve the desired amount of M^{O}A₂₀₀L₃₀ (e.g. 6 wt %), resulting in a viscous solution. Separately, the anionic diblock copolypeptide, M^{O}A₁₅₀E₅₅, was dissolved in an equal volume of 1×PBS at the desired concentration (e.g. 5 wt %). The anionic copolypeptide solution was then added to the viscous cationic copolypeptide solution, and the resulting mixture vortexed for 20 seconds leading to DCH_{DN} hydrogel formation (e.g. 3 wt % DCH_{MO} and 5 wt % DCH_{PIC}) within 5-30 seconds depending on copolypeptide concentrations and compositions.

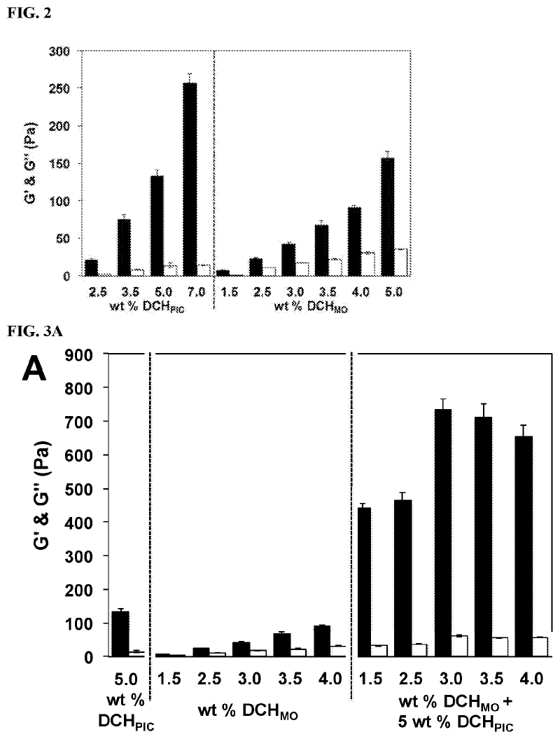

FIG. 2 shows the storage modulus, G′ (Pa, black), and loss modulus, G″ (Pa, white), of individual DCH_{PIC} and DCH_{MO} hydrogel components at different concentrations in 1×PBS buffer at 25° C. All G′ and G″ values were measured at an angular frequency of 5 rad/s and a strain amplitude of 0.01.

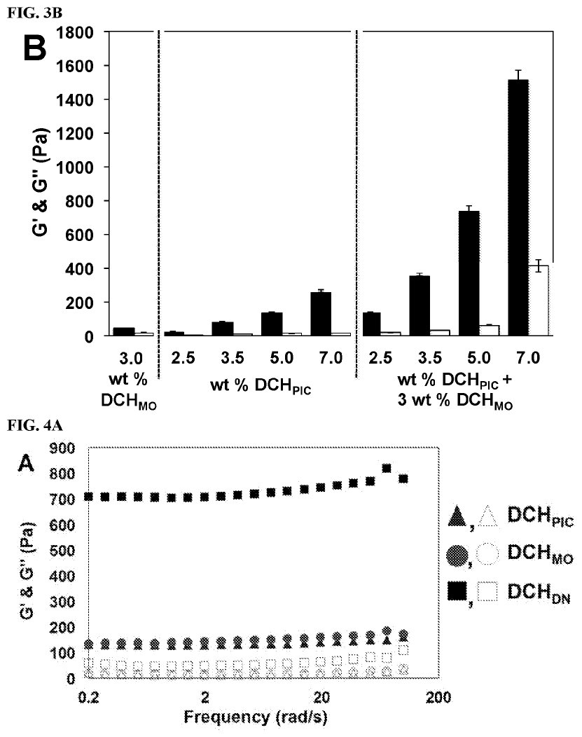

FIG. 3A shows G′ (Pa, black) and G″ (Pa, white) of DCH_{DN} composed of 5 wt % DCH_{PIC} and varying concentrations of DCH_{MO} in 1×PBS buffer at 25° C. Data for individual 5 wt % DCH_{PIC} and DCH_{MO} hydrogel components at different concentrations in 1×PBS buffer at 25° C. are included for reference.

FIG. 3B shows G′ (Pa, black), and loss modulus, G″ (Pa, white), of DCH_{DN} composed of 3 wt % DCH_{MO} and varying concentrations of DCH_{PIC} in PBS buffer at 25° C. Data for individual 3 wt % DCH_{MO} and DCH_{PIC} hydrogel components at different concentrations in 1×PBS buffer at 25° C. are included for reference. All G′ and G″ values in FIGS. 3A-3B were measured at an angular frequency of 5 rad/s and a strain amplitude of 0.01.

### Example 3: Rheology Measurements on Copolypeptide Hydrogels

An Anton Paar Instruments MCR 302 rheometer with a 25 mm diameter and 1° cone plate geometry and solvent trap was used for all measurements. Frequency sweeps were measured at constant strain amplitude of 0.01. Strain sweeps were measured at a constant frequency of 5 rad/s. All measurements were repeated 3 times for each hydrogel sample and the results were averaged and plotted. To evaluate shear thinning and recovery behavior of DCH, the strain amplitude was stepped from 0.01 to 10, maintained at 10 for 2 min and then returned to 0.01 to evaluate the recovery of mechanical properties at a fixed frequency of 5 rad/s.

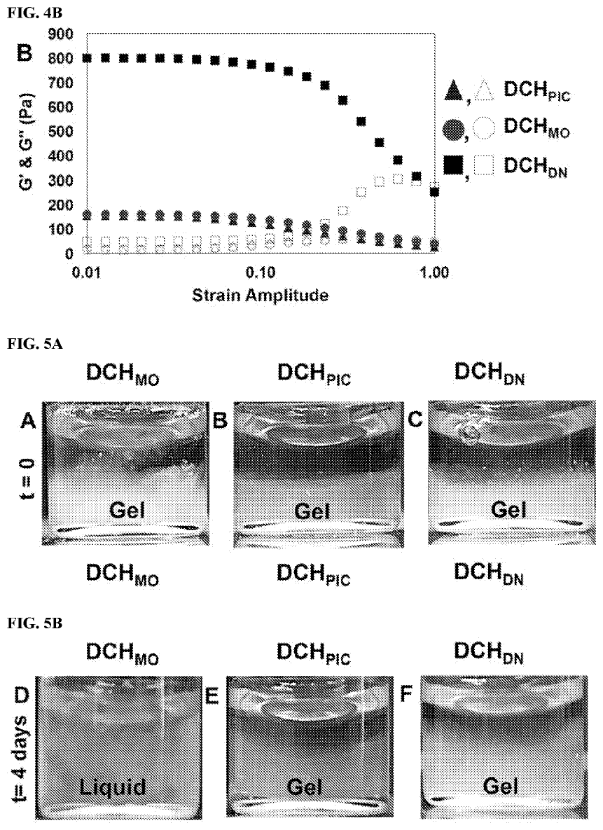

FIGS. 4A-4B shows rheology data for 5 wt % DCH_{PIC}, 5 wt % DCH_{MO}, and DCH_{DN} (3 wt % MO+5 wt % PIC) hydrogels in 1×PBS buffer at 20° C. (A) G′ (Pa, solid symbols) and G″ (Pa, open symbols) of hydrogel samples as functions of angular frequency at constant strain amplitude of 0.01. (B) Storage modulus, G′ (Pa, solid symbols), and loss modulus, G″ (Pa, open symbols), of hydrogel samples as functions of strain amplitude at a constant frequency of 5 rad/s.

### Example 4: Hydrogel Swelling Measurements

Hydrogels of 6 wt % DCH_{PIC}, 6 wt % DCH_{MO}, and DCH_{DN} (3 wt % MO and 5 wt % PIC) were prepared in 2 ml scintillation vials and allowed to stand for 1 hr. DMEM cell culture media was then placed on top of each hydrogel sample and samples were stored in a refrigerator (0° C.) for different periods of time. At each time point, the supernatant liquid was pipetted out of each sample without disturbing the gel at the bottom. The supernatant volumes were subtracted from the original media volume to determine swelling ratios. The hydrogel samples were also subjected to inversion tests to verify hydrogel integrity. Finally, the supernatant liquid was replaced on top of each hydrogel and incubation of samples allowed to continue.

FIGS. 5A-5I show stability of diblock copolypeptide hydrogels against dilution. Equal volume samples of DCH_{MO} (5.0 wt %), DCH_{PIC} (5.0 wt %), and DCH_{DN} (3 wt % DCH_{MO} and 5 wt % DCH_{PIC}) in 1×PBS were each diluted with an equal volume of DMEM cell culture media. (A-C) Layers of cell media formed over all hydrogels at the beginning of the experiment (time 0). (D-F) After 4 days, the DCH_{PIC} and DCH_{DN} hydrogels remained intact while DCH_{MO} had dispersed into the full volume of media and was a liquid. (G-I) After 7 days, both DCH_{PIC} and DCH_{DN} remained intact as hydrogels underneath the media.

### Example 5: Quantification of Sample Loss During Hydrogel Swelling

Samples of DCH_{PIC} (5.0 wt %) and DCH_{DN} (3 wt % DCH_{MO} and 5 wt % DCH_{PIC}) were prepared in 1×PBS and were each diluted with an equal volume of 1×PBS and let stand for 7 days. At this time, all supernatant liquid was removed from each sample and lyophilized. Recovered polypeptide from the supernatants was weighed, and the contribution from PBS salts removed. It was found that 88 wt % and 81 wt % of diblock copolypeptides were retained in DCH_{PIC} and DCH_{DN}, respectively.

### Example 6: Normalized Swelling Ratio Measurements

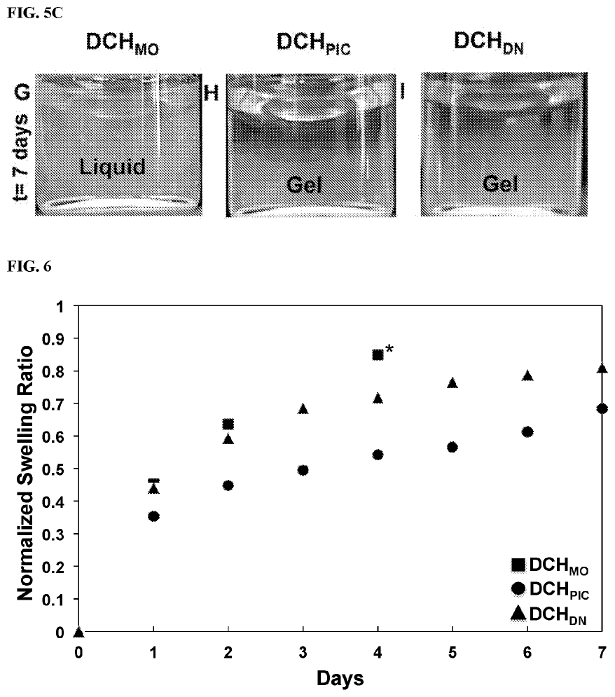

Equal volume samples of DCH_{MO} (5.0 wt %), DCH_{PIC} (5.0 wt %), and DCH_{DN} (3 wt % MO and 5 wt % PIC) in 1×PBS were each diluted with an equal volume of DMEM cell culture media. Hydrogel swelling was monitored by removal of all supernatant liquid above each hydrogel at different time points (FIG. 6). Normalized swelling ratio was calculated as: (weight of sample after swelling−weight of initial sample)/weight of initial sample. *DCH_{MO} was no longer a hydrogel by day 4.

### Example 7: Mechanical Recovery of Diblok Copolypeptide Hydrogels

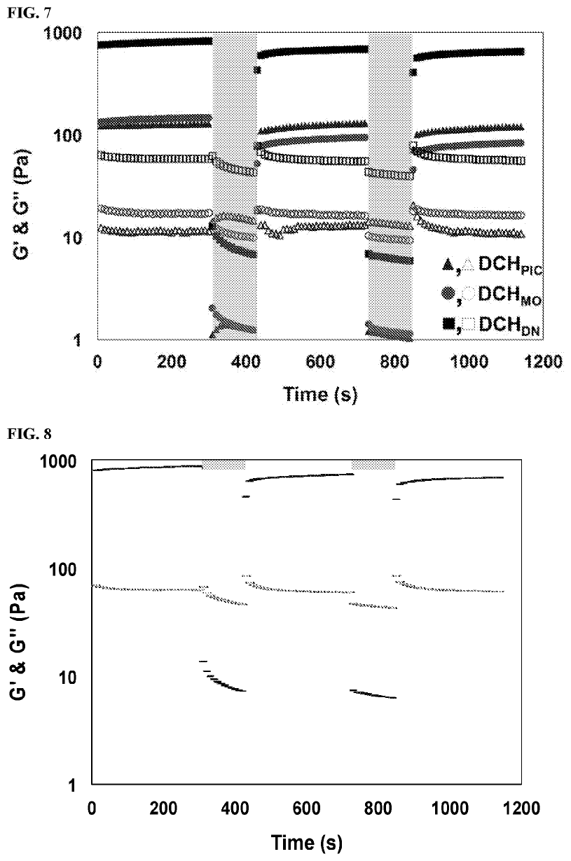

FIG. 7 shows mechanical recovery of 5 wt % DCH_{PIC}, 5 wt % DCH_{MO} and DCH_{DN} (3 wt % DCH_{MO}+5 wt % DCH_{PIC}) over time in 1×PBS buffer at 25° C. (G′=solid symbols; G″=open symbols) after application of stepwise large-amplitude oscillatory breakdown (gray regions=strain amplitude of 10 at 10 rad/s for 120 s) followed by low-amplitude linear recovery (white regions=strain amplitude of 0.01 at 5 rad/s for 300 s).

FIG. 8 shows mechanical recovery of DCH_{DN} (3 wt % DCH_{MO}+5 wt % DCH_{PIC}) over time in 1×PBS buffer at 25° C. (G′=solid symbols; G″=open symbols) after application of stepwise large-amplitude oscillatory breakdown (gray regions=strain amplitude of 10 at 10 rad/s for 120 s) followed by low-amplitude linear recovery (white regions=strain amplitude of 0.01 at 5 rad/s for 300 s).

### Example 8: Fluorescent Probe Conjugation to (MOA)155E55 and (MOA)200L30 Copolypeptides

Alexa Fluor 488 NHS ester (AF 488 NHS) was conjugated to the N-terminal amine groups of (M^{O}A)₁₅₅E₅₅. (M^{O}A)₁₅₅E₅₅ (10 mg) was dissolved in DI water (pH 7) (1 ml) in a scintillation vial (20 ml). AF 488 NHS was dissolved in DI water (pH 7) (1 mg/ml) and added to the 1% (w/v) copolypeptide solution at a 1.25:1 molar ratio of fluorescent probes per copolypeptide chain. The reaction was allowed to proceed for 24 h at ambient temperature. After AF 488 modification, the resulting solution was dialyzed (2000 MWCO) against DI water for 2 d, and then freeze-dried to yield the product as a yellow solid. Alexa Fluor 633 NHS ester (AF 633 NHS) was conjugated onto the N-terminal amine groups of (M^{O}A)₁₅₀L₃₀ using a similar procedure.

### Example 9: Laser Scanning Confocal Microscopy (LSCM) of Fluorescently Labeled Hydrogels

LSCM images of hydrogels were taken on a Leica TCS-SP8 MP-Inverted confocal and multiphoton microscope equipped with an argon laser (476 and 488 nm blue lines), a diode (DPSS) laser (561 nm yellow-green line), and a helium-neon laser (633 nm far red line). Fluorescently labeled hydrogel samples were visualized on glass slides with a spacer between the slide and the cover slip (double-sided tape) allowing the self-assembled structures to be minimally disturbed during focusing. An optical section of 0.896 μm was used. Leica LAS-X software was used for 3D rendering. The resulting 3D model was processed via histogram equalization. Sample imaging was performed at the Advanced Light Microscopy-Spectroscopy Center (ALMS) at the UCLA California NanoSystems Institute (CNSI).

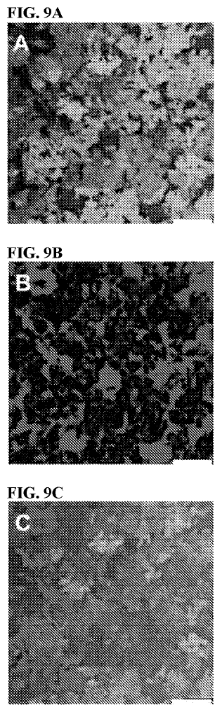

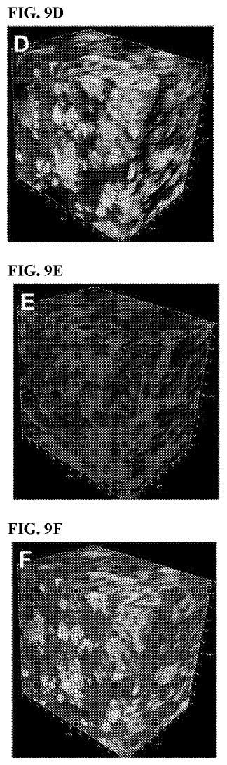

FIGS. 9A-9F show laser scanning confocal microscopy (LCSM) images of DCH_{DN} (3 wt % DCH_{MO}+5 wt % DCH_{PIC}). (A-C) LSCM images of DCH_{DN} prepared using Alexa Fluor 488 labeled (M^{O}A)₁₅₅E₅₅ and Alexa Fluor 633 labeled (M^{O}A)₂₀₀L₃₀ components that show separate interpenetrating networks of DCH_{MO} (red) and DCH_{PIC} (green) (z-thickness=0.896 μm). (A) Alexa Fluor 488 channel, (B) Alexa Fluor 633 channel, (C) merged image of (A) and (B). (D-F) 3D renderings of DCH_{DN} z-slice stacks showing separate interpenetrating networks of DCH_{MO} (red) and DCH_{PIC} (green). (D) Alexa Fluor 488 channel, (E) Alexa Fluor 633 channel, (F) merged image of (D) and (E). All scale bars=10 μm.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims

1. A composition comprising a first copolypeptide comprising Substructure I, a second copolypeptide comprising Substructure II, a third copolypeptide comprising Substructure III, and water, wherein Substructure I is depicted as follows:
—Xₘ—Cₚ— Substructure I;
Substructure II is depicted as follows:
—Yₙ—A_{q}— Substructure II;
Substructure III is depicted as follows:
—Zᵣ—Dₜ— Substructure III;
each instance of X is an amino acid residue independently selected from a non-ionic, hydrophilic amino acid, sarcosine, glycine, and alanine;
each instance of Y is an amino acid residue independently selected from a non-ionic, hydrophilic amino acid, sarcosine, glycine, and alanine;
each instance of Z is an amino acid residue independently selected from a non-ionic, hydrophilic amino acid, sarcosine, glycine, and alanine;
in at least 20% of the instances of C, C is an amino acid residue independently selected from a cationic, hydrophilic amino acid;
in at least 20% of the instances of A, A is an amino acid residue independently selected from an anionic, hydrophilic amino acid;
in at least 20% of the instances of D, D is an amino acid residue independently selected from a non-ionic, hydrophobic amino acid;
m is about 100 to about 600;
n is about 100 to about 600;
r is about 100 to about 600;
p is about 20 to about 200;
q is about 20 to about 200;
t is about 10 to about 200;
at least 90 mol % of the C amino acid residues are (D)-amino acid residues or at least 90 mol % of the C amino acid residues are (L)-amino acid residues;
at least 90 mol % of the A amino acid residues are (D)-amino acid residues or at least 90 mol % of the A amino acid residues are (L)-amino acid residues; and
at least 90 mol % of the D amino acid residues are (D)-amino acid residues or at least 90 mol % of the D amino acid residues are (L)-amino acid residues;
the first copolypeptide and the second copolypeptide are not covalently linked to the third copolypeptide;
the total concentration of the first copolypeptide and the second copolypeptide is about 1% to about 15%; and
the concentration of the third copolypeptide is about 1% to about 10%.

2. The composition of claim 1, wherein the composition comprises a first copolypeptide comprising Substructure I′, a second copolypeptide comprising Substructure II′, a third copolypeptide comprising Substructure III′, and water, wherein Substructure I is depicted as follows:
—Xₘ—Cₚ— Substructure I′;
Substructure II is depicted as follows:
—Yₙ—A_{q}— Substructure II′;
Substructure III is depicted as follows:
—Zᵣ—Dₜ— Substructure III′;
each instance of X is an amino acid residue independently selected from a non-ionic, hydrophilic amino acid, glycine, and alanine;
each instance of Y is an amino acid residue independently selected from a non-ionic, hydrophilic amino acid, glycine, and alanine;
each instance of Z is an amino acid residue independently selected from a non-ionic, hydrophilic amino acid, glycine, and alanine;
in at least 20% of the instances of C, C is an amino acid residue independently selected from a cationic, hydrophilic amino acid;
in at least 20% of the instances of A, A is an amino acid residue independently selected from an anionic, hydrophilic amino acid;
in at least 20% of the instances of D, D is an amino acid residue independently selected from a non-ionic, hydrophobic amino acid;
m is about 100 to about 600;
n is about 100 to about 600;
r is about 100 to about 600;
p is about 20 to about 200;
q is about 20 to about 200;
t is about 10 to about 200;
at least 90 mol % of the C amino acid residues are (D)-amino acid residues or at least 90 mol % of the C amino acid residues are (L)-amino acid residues;
at least 90 mol % of the A amino acid residues are (D)-amino acid residues or at least 90 mol % of the A amino acid residues are (L)-amino acid residues; and
at least 90 mol % of the D amino acid residues are (D)-amino acid residues or at least 90 mol % of the D amino acid residues are (L)-amino acid residues;
the first copolypeptide and the second copolypeptide are not covalently linked to the third copolypeptide;
the total concentration of the first copolypeptide and the second copolypeptide is about 1% to about 15%; and
the concentration of the third copolypeptide is about 1% to about 10%.

3. (canceled)

4. The composition of claim 1, wherein each instance of X is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide.

5-7. (canceled)

8. The composition of claim 1, wherein at least 85 mol % of the X amino acid residues are methionine sulfoxide.

9-11. (canceled)

12. The composition of claim 1, wherein each instance of Y is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, and homomethionine sulfoxide.

13-15. (canceled)

16. The composition of claim 1, wherein at least 85 mol % of the Y amino acid residues are methionine sulfoxide.

17-21. (canceled)

22. The composition of claim 1, wherein each instance of C is lysine, ornithine, or arginine.

23-27. (canceled)

28. The composition of claim 1, wherein each instance of A is glutamic acid or aspartic acid.

29-31. (canceled)

32. The composition of claim 1, wherein each instance of Z is an amino acid residue independently selected from sarcosine, glycine, alanine, methionine sulfoxide, S-alkyl-cysteine sulfoxide, S-alkyl cysteine sulfone, S-alkyl-homocysteine, S-alkyl-homocysteine sulfoxide, glycosylated cysteine, serine, homoserine, homomethionine sulfoxide.

33-35. (canceled)

36. The composition of claim 1, wherein at least 85 mol % of the Z amino acid residues are methionine sulfoxide.

37-41. (canceled)

42. The composition of claim 1, wherein each instance of D is leucine, alanine, or phenylalanine.

43. (canceled)

44. (canceled)

45. The composition of claim 1, wherein m is about 100, about 110, about 120, about 130, about 140, about 150, about 160, about 170, about 180, about 190, about 200, about 210, or about 220; n is about 100, about 110, about 120, about 130, about 140, about 150, about 160, about 170, about 180, about 190, about 200, about 210, or about 220; r is about 100, about 110, about 120, about 130, about 140, about 150, about 160, about 170, about 180, about 190, about 200, about 210, or about 220; p is about 20, about 30, about 40, about 50, about 60, about 70, about 80, about 90, or about 100; q is about 20, about 30, about 40, about 50, about 60, about 70, about 80, about 90, or about 100; and t is about 10, about 20, about 30, about 40, about 50, about 60, about 70, about 80, about 90, or about 100.

46-53. (canceled)

54. The composition of claim 1, wherein the polydispersity of the first copolypeptide and the second copolypeptide is less than 1.5.

55. (canceled)

56. The composition of claim 1, wherein the number of amino acid residues in the first copolypeptide is from about 90% to about 110% of the number of amino acid residues in the second copolypeptide.

57. The composition of claim 1, wherein the composition comprises (M^{O}A)₁₅₅E₃₀, (M^{O}A)₁₅₅E₆₀, (M^{O}A)₁₅₅E₉₀, (M^{O}A)₁₅₅E₁₂₀, (M^{O}A)₁₅₅(rac-E)₆₀, (M^{O}A)₁₅₅K₃₀, (M^{O}A)₁₅₅K₆₀, (M^{O}A)₁₅₅K₉₀, (M^{O}A)₁₅₅K₁₂₀, (M^{O}A)₁₅₀E₅₅, (M^{O}A)₁₅₀K₅₅, (M^{O}A)₁₅₀L₂₀, (M^{O}A)₁₅₀L₃₀; or a combination of the foregoing.

58-65. (canceled)

66. The composition of claim 1, wherein the total concentration of the first copolypeptide and the second copolypeptide in the composition is about 5.0 wt. %, and the concentration of the third copolypeptide in the composition is about 2.5 wt. %.

67. (canceled)

68. The composition of claim 1, wherein the molar ratio of C to A is from about 0.95 to about 1.05; the molar ratio of X to Y is from about 0.95 to about 1.05; and the molar ratio of D to A is from about 0.4 to about 0.6.

69. (canceled)

70. (canceled)

71. The composition of claim 1, wherein the composition further comprises a salt, a buffer, or a plurality of cells.

72-76. (canceled)

77. A method of making the composition of claim 1, the method comprising:
dissolving the first copolypeptide in an aqueous medium;
separately adding the third copolypeptide to the aqueous medium to form a mixture; and
mixing the mixture with a solution of the second copolypeptide, thereby forming the composition; or
dissolving the second copolypeptide in an aqueous medium;
separately adding the third copolypeptide to the aqueous medium to form a mixture; and
mixing the mixture with a solution of the first copolypeptide, thereby forming the composition.

78-83. (canceled)

84. A method of delivering a drug to a biological target using the composition of claim 1, the method comprising:
dissolving the drug in a first aqueous medium;
dissolving the first copolypeptide in the first aqueous medium, to form a second aqueous medium;
separately adding the third copolypeptide to the second aqueous medium, to form a mixture;
mixing the mixture with a solution of the second copolypeptide, thereby forming the composition encapsulating the drug; and
contacting the biological target with the composition; or
dissolving the drug in a first aqueous medium;
dissolving the second copolypeptide in the first aqueous medium, to form a second aqueous medium;
separately adding the third copolypeptide to the second aqueous medium, to form a mixture;
mixing the mixture with a solution of the first copolypeptide, thereby forming the composition encapsulating the drug; and
contacting the biological target with the composition.

85-91. (canceled)
